(19) United States
(12) Patent Application Publication
(10) Pub. No.: US 2014/0356934 A1
(43) Pub. Date: Dec. 4, 2014

(54) Incubation Chamber

(71) Applicant: SunChrom GmbH, Friedrichsdorf (DE)

(72) Inventors: Günes BARKA, Friedrichsdorf (DE); Dominic BÄUMLISBERGER, Oberursel (DE)

(73) Assignee: SunChrom GmbH, Friedrichsdorf (DE)

(21) Appl. No.: 14/288,858

(22) Filed: May 28, 2014

(30) Foreign Application Priority Data
May 31, 2013 (DE) 10 2013 009 136.5

(51) Int. Cl. C12M 1/00; C12M 1/02; C12M 1/12

(52) U.S. Cl. CPC C12M 41/14; C12M 25/06; C12M 41/24; C12M 23/38
USPC 435/286.1

(57) ABSTRACT

An incubation chamber (10) having a water reservoir (12) has a climate controlling device, which makes it possible to set a desired temperature, as well as an object holder (18) for biological or chemical specimens. To make defined and replicable conditions possible, for instance for digestion processes of proteins, it is proposed that the climate controlling device has two separate climate zones (14, 16), which can be regulated separately from one another, a first climate zone (14) being located in the vicinity of the cover of the chamber (10).

### FIELD OF THE INVENTION

The present invention relates to an incubation chamber having a moisture reservoir, a climate controlling device that makes it possible to set a desired temperature, and a holder for biological or chemical specimens.

### BACKGROUND OF THE INVENTION

Incubation chambers, also known as incubation cupboards, are used to cultivate bacteria, other microorganisms and target lines on suitable media, or in other words as a rule to propagate them. They can also serve to run digestion processes of proteins or larger peptides into smaller peptides or parts of the starting material, by means of certain proteolytic enzymes, such as trypsin, chemotrypsin, elastase, and so forth, under replicable conditions. The smaller peptide molecules have entirely different chemical properties and can be much more easily manipulated, so that their structure is easier to determine.

If proteins are digested enzymatically, and the resultant peptides are analyzed by chemical or other physical-chemical methods and their amino acid sequences are determined, then the protein primary sequences can be determined by means of various techniques. In proteome analysis, the term so-called bottom-up batch is also used. For comprehensive analysis of the primary sequences of proteins, this method represents the state of the art.

The digestion enzymes, in terms of their structure, are themselves proteins or protein-like compounds. Like all proteins, they are highly vulnerable to chemical or physical changes in their surroundings. Many lose their activities if they dry out, are overheated, or come into contact with organic solvents. The digestion of proteins is done in solution, in gel matrices, on membranes, or in conjunction with imaging mass spectrometry, in tissue slices. This requires meeting certain prerequisites, such as the nature of the solution or solvent, the pH value, the salt content, the temperature, and possibly other peripheral conditions.

The structural and sequential clarification of proteins is the primary prerequisite for recognizing their function in the living organism. That requires not only knowing the sequence of the amino acid building blocks. These properties are today investigated and clarified by means of various mass spectrometry techniques. However, besides peptides, the still-intact proteins can also be analyzed by mass spectrometry, in order to determine the precise molecular weight and recognize any possible degradations.

In recent years, imaging mass spectrometry has become established; which makes it possible to recognize and determine/detect proteins directly, without first having to remove them by extraction from their original surroundings. Among others, various ionization methods are used, such as MALDI (Matrix-Assisted Laser Desorption/Ionization), DESI (Desorption Electrospray Ionization), and SIMS (Secondary Ion Mass Spectrometry). This method has the great advantage that with it, the local distribution of the individual proteins can be detected, and the local association with certain regions of a tissue, for instance in kidneys, the liver or the brain, takes appropriate account of the wish to learn more details about the function of the proteins.

Mass analysis of intact proteins directly from tissues is markedly more difficult, compared to smaller molecules. In contrast to proteins, the peptides can be better extracted from tissues, incorporated into matrix crystals, and ionized in the mass spectrometer. However, in order to leave the peptide that results from a protein by enzymatic digestion in the original site in the tissue, certain criteria must be met during the digestion process.

First, it is important that the digestion be performed within a defined temperature range, which as a rule is between 25 and 50° C., because for most enzymes, the ideal reaction temperature with the maximum reaction output and minimum loss of activity is located in this temperature range. Water is necessary for the digestion process, but too much water on or in the tissue causes the peptides to leave their original site and diffuse in all directions. This leads to a loss of local resolution and thus of the possibility of obtaining separate information of areas located close together in a tissue, so that the valuable information associated with that is lost.

In order to ensure this, the water content in the tissue must be constant and easily condensed, but neither too high nor too low. Ideally, saturated moisture is present not only the surface but also in deeper regions of the tissue, which is sufficient for an enzymatic reaction over a relatively long time, up to several hours.

There are publications which propose incubation chambers that are created for the particular application on the order of a temporary version, but sometimes it is proposed that incubators be dispensed with entirely, by using special techniques for enzyme addition. In the known incubation chambers, less has been reported about the yields of the digestion processes, but directly or indirectly problems are reported that relate to the adherence to the aforementioned peripheral conditions. Water condensation in the incubation chamber is also a major problem, since dripping or flowing water droplets can flood the tissue.

### BRIEF SUMMARY OF THE INVENTION

The object of the present invention is to create an incubation chamber which ensures the course of the digestion processes mentioned under defined and replicable conditions.

According to the invention, the object is attained by an incubation chamber of the type defined at the outset in which the climate controlling device has two separate climate zones, which can be regulated separately from one another, and the first climate zone is located in the vicinity of the cover of the incubation chamber.

It has been found that by locating a climate zone in the vicinity of the cover, the peripheral reactions under which a digestion process, for instance, is to proceed can be more easily maintained, and the separate temperature regulation in the vicinity of the cover aids in reliably avoiding the formation there of droplets as a consequence of water condensation. Water droplets dropping onto the tissue from the cover are thus no longer any problem. By maintaining an optimal temperature, the digestion processes are optimized, and an optimal outcome can be achieved in minimal time. Location information in tissue specimens can be reliably obtained.

In a preferred embodiment of the invention, it is provided that at least one of the two climate zones also has a cooling function. In this way, it is possible to cool the temperature in the chamber down, for instance to 4° C., which proves to be advantageous as a temperature for object holding and for the object carriers and specimen carriers held therein for applying enzymes onto the specimens. After the incubation chamber has been heated for the enzymatic digestion, the incubation chamber can then also be simultaneously cooled slowly again, without resulting in unwanted water condensation inside the incubation chamber, which could occur especially on the specimens, such as the tissue slices located there.

In a further preferred embodiment of the invention, it is provided that the second climate zone, which preferably also makes the cooling function possible, is located in the bottom plate of the incubation chamber. This second climate zone can also be embodied as stronger in terms of its performance, since it can be available primarily for establishing a certain temperature, for instance of up to 60° C., in the incubation chamber, while the first climate zone in the cover, by suitable tempering of the inner side of the cover, primarily counteracts the formation of condensate drops at that point.

The moisture reservoir of the incubation chamber is preferably embodied as a tub, whose active surface area is increased by means of a felt or foam layer or other water-storing materials. This tub can, like the object holder, comprise thermally conductive metal and/or plastic, and the object holder and the tub can also be embodied in one piece. The object holder is designed such that the object carrier and/or specimen carrier is kept at a defined spacing from the moisture reservoir. Optionally, the holder can be such that the object carrier or specimen carrier can be disposed selectively, with specimens located downward toward or closest to the reservoir or upward toward or closest to the free space since the specimens are typically adhesively held in place.

In an especially preferred embodiment of the invention, a fan apparatus is provided. A fan apparatus offers the advantage that by purposeful circulation of air in all regions of the chamber, homogeneous temperature and moisture properties can be furnished, and the evaporation speed can be varied. Preferably, the fan power can be adjusted to between 0 and a maximum value, since it can be used to adjust the ambient conditions. For instance, if the measured humidity is too high, the fan power can be increased in order to reduce the processes of condensation in the vicinity of the active specimen surface.

Preferably, temperature and/or moisture sensors are provided in the incubation chamber; and via a control device, they control the heating output and/or the fan power as a function of the measured values. In this way, all the parameters can be varied in real time, and defined specified values, such as the temperature difference between the first and second climate zones, can be stored in memory beforehand.

The cover of the incubation chamber should be removable or at least capable of being pivoted upward to be removed out of the way, for the sake of accessibility.

### BRIEF DESCRIPTION OF THE DRAWINGS

One exemplary embodiment of the invention will now be described in further detail, in conjunction with the accompanying drawings. In the drawings:

FIG. 1 is a sectional elevation view of an incubation chamber;

FIG. 2 is a sectional elevation view in the plane of the fan;

FIG. 3 is a sectional elevation through the incubation chamber, rotated relative to FIG. 1; and

FIG. 4 is a sectional top view of the incubation chamber in the plane of the object carrier holder.

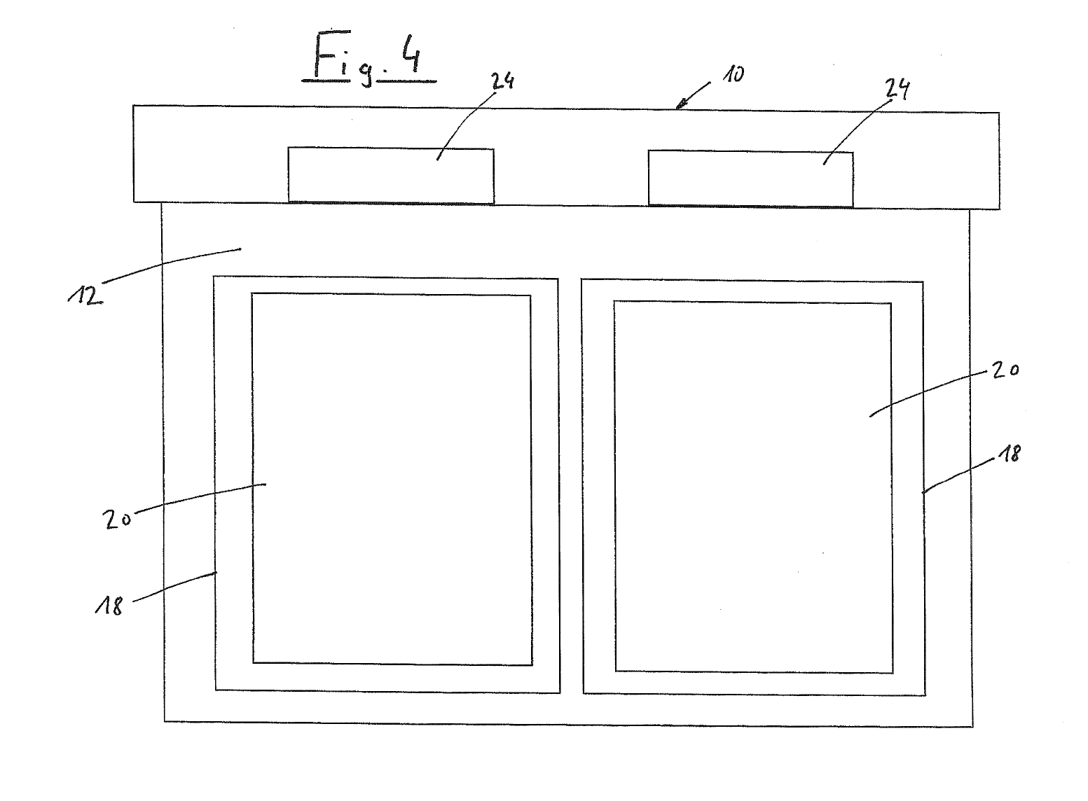

### DETAILED DESCRIPTION OF THE INVENTION

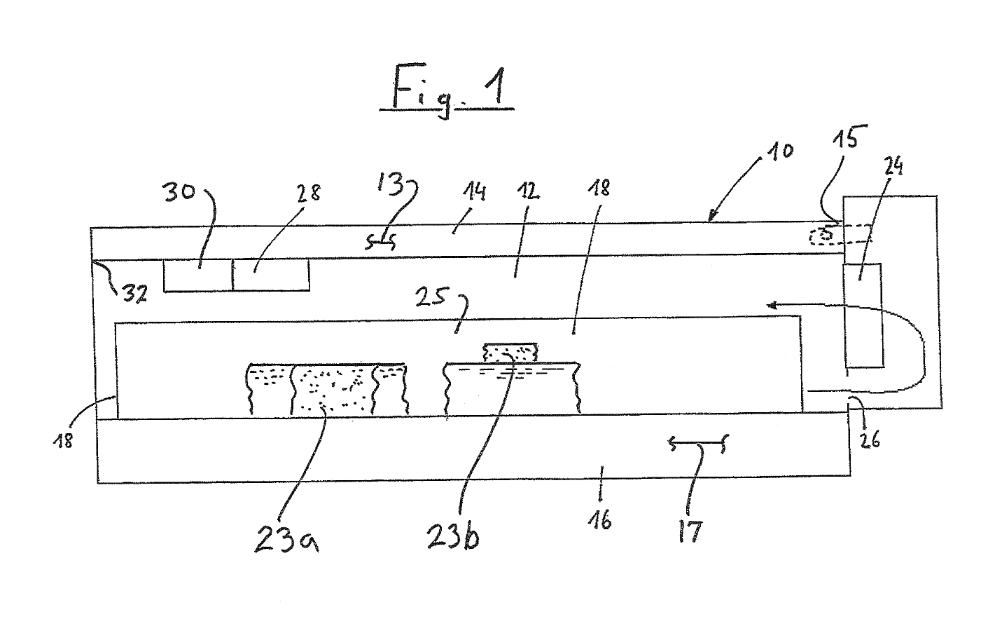

In FIG. 1, an incubation chamber 10 is shown, which has a chamber space 12 that is surrounded on all sides. Electric heating elements 13 (not shown individually) for creating a first climate zone are provided in a removable cover 14. In a bottom plate 16, Peltier elements 17 are provided, which are used as cooling or heating elements for creating a second climate zone. In the chamber space 12, there is an object carrier holder 18, on which object carriers 20 or MALDI specimen carriers or other carriers can be disposed.

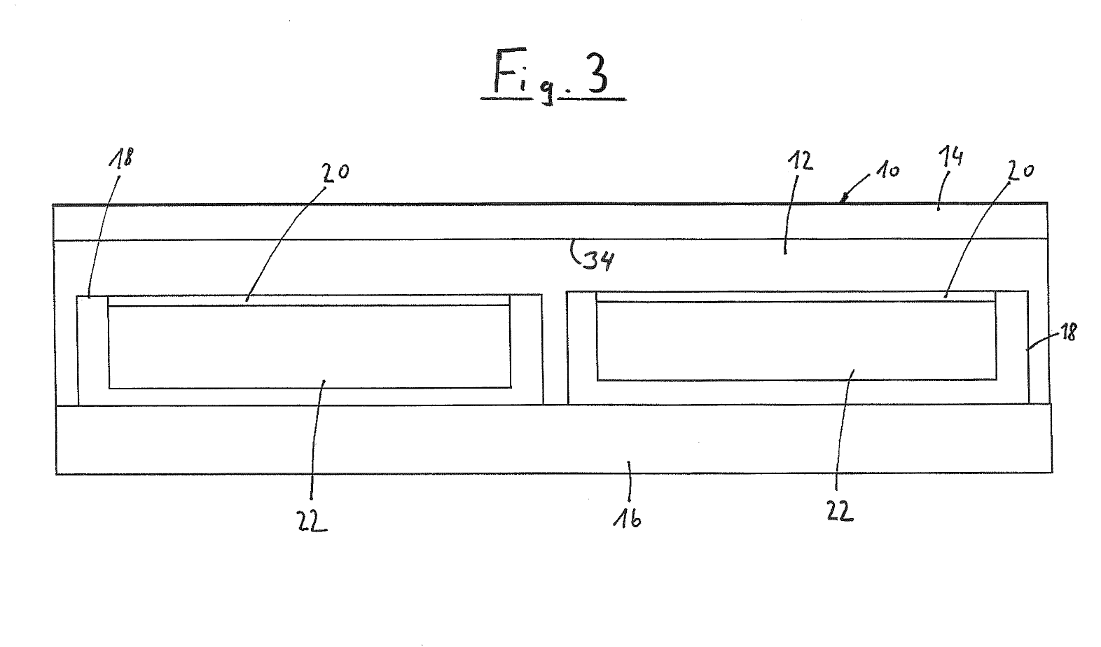

The object holder 18 is, as can readily be seen from FIG. 3, embodied as a water reservoir 22; this water reservoir is filled with distilled water or mixtures of water, so that it forms the necessary moisture reservoir for creating the humidity. To increase the effective surface area, at least a portion of the water is provided in a water-absorbing and -storing layer 23a, for instance of felt or foam or other suitable materials. A corresponding layer 23b can also rest on the water. Between the felt or foam layer 23a and the object carriers 20, a space or interstice 25 remains, so that direct contact with the object carrier is precluded. In FIG. 3, removable cover 14 is hinged at 34 to be removed out of the way rather than being completely removable out of the way as in FIG. 1.

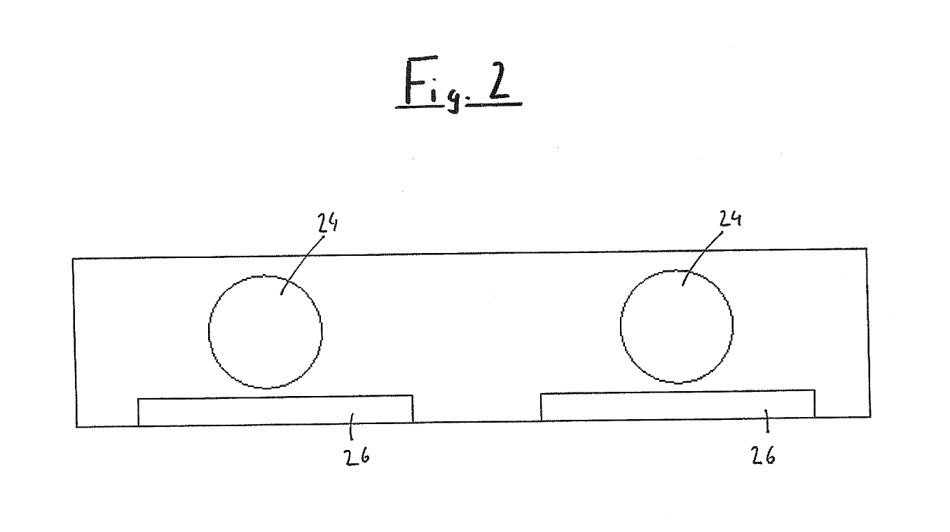

The remaining interstice 25 between the object carrier 20 and the felt or foam layer 23a is also necessary for the air circulation, which is illustrated in FIGS. 1 and 3. In the exemplary embodiment shown, two fans 24 are provided on the back side of the incubation chamber 10, which extract air through air ducts 26 (see FIG. 2) from the interstice 25 between the object carrier 20 and the water reservoir 22 and blow it into the region between the heatable cover plate 14 and the object carrier 20. In this way, the process of evaporation is favored, and a more uniform moisture content in the entire interior of the chamber space 12 is ensured.

By means of the air saturated with moisture, which is moved directly past the object carriers 20, or the tissue slices mounted on them and coated with enzymes, an atmosphere that is constantly saturated with water is achieved, which is definitive of an adequate, replicable outcome of digestion. By means of the heatable cover plate 14, it is ensured that condensate formation on the underside of the cover 14 is precluded, so that there is no risk that condensate drops can drip onto the object carriers 20, which could impair the digestion process and at least the local information of the specimen slices. A moisture sensor 28 in the chamber interior constantly monitors the moisture, so that it can be kept within the range of saturation around 100%. A temperature sensor 30 serves to set the desired temperature, and the incubation chamber 10 enables heating up to 60° C., since it has been demonstrated that the digestion processes in some enzymes proceed markedly faster at reaction temperatures over 37° C.

The cooling function has the advantage that the object carrier, during the application of the enzymes in a plurality of layers, can be kept below the ambient temperature, so that the enzymes can better penetrate the tissue before the digestion process is then started at a defined time.

The incubation chamber 10 is closed by means of a suitable seal 32, in order on the one hand to prevent unnecessary losses of moisture and to ensure secure partitioning off from the atmosphere located outside the incubation chamber 10 on the other.

The temperature can also be measured at a plurality of points inside the incubation chamber 10 and can also be regulated separately by means of the two climate zones. The entire course of a digestion process can be logged and later printed out by the controlling electronics, so that the user obtains valuable information which can be helpful in optimizing the digestion parameters for certain applications or for making the desired universal replicability of the digestion process possible. The temperature of the heatable cover plate 14 can be located under active control above the temperature of the heated bottom plate 16, to reliably avoid condensation processes on the underside.

1. An incubation chamber having a moisture reservoir, a climate controlling device which enables a desired temperature to be set, and an object holder for biological or chemical specimens, characterized in that the climate controlling device has two separate climate zones which are regulatable separately from one another, and the first climate is located in the vicinity of the cover of the chamber.

2. The incubation chamber of claim 1, characterized in that at least one of the two climate zones also has a cooling function.

3. The incubation chamber of claim 1, characterized in that the second climate zone is located in the base plate of the incubation chamber.

4. The incubation chamber of claim 1, characterized in that a moisture reservoir is embodied by a tub, an active surface area of which is increased by a layer of felt or foam.

5. The incubation chamber of claim 4, characterized in that the object holder and the tub comprise heat-conducting metal and/or plastic.

6. The incubation chamber of claim 1, characterized in that the climate controlling device is designed such that a temperature in the chamber of between 4° C. and 60° C. can be set.

7. The incubation chamber of claim 4, characterized in that the object holder is embodied for receiving object carriers and/or MALDI specimen carriers, and the object carriers and/or specimen carriers are held at a defined spacing from the moisture reservoir.

8. The incubation chamber of claim 7, characterized in that the specimens on the object carriers or specimen carriers can be located selectively downward toward the moisture reservoir or upward toward the free space.

9. The incubation chamber of claim 1, characterized in that a fan apparatus is provided, with which the air inside the incubation chamber can be put into circulation along the object carriers or specimen carriers.

10. The incubation chamber of claim 9, characterized in that the power of the fan apparatus can be regulated between 0 and a maximum value.

11. The incubation chamber of claim 1, characterized in that temperature and/or moisture sensors are provided in the chamber space, which control the temperature and/or the humidity in the chamber space as a function of the measured values, via a control device.

12. The incubation chamber of claim 1, characterized in that the cover of the chamber is removable or can be pivoted upward.

13. The incubation chamber of one of claim 1, characterized in that the interior of the chamber space is sealed off from the surroundings by a closed, encompassing seal.
